(19) United States
(12) Patent Application Publication
(10) Pub. No.: US 2012/0237502 A1
(43) Pub. Date: Sep. 20, 2012

(54) METHOD FOR TREATING BREAST CANCER AND OVARIAN CANCER

(75) Inventor: James W. Darnowski, Barrington, RI (US)

(73) Assignee: Takeda Pharmaceutical Company Limited, Osaka-shi (JP)

(21) Appl. No.: 13/299,372

(22) Filed: Nov. 18, 2011

Related U.S. Application Data
(60) Provisional application No. 61/415,097, filed on Nov. 18, 2010.

(51) Int. Cl. A61K 31/4188; A61K 31/513; A61K 31/675; A61P 35/00; A61K 33/24; A61K 39/395; A61K 31/7068; A61K 31/58; A61K 31/704

(52) U.S. Cl. 424/133.1; 514/176; 514/393; 514/274; 514/110; 514/34; 424/649; 514/49; 514/171; 514/170

(57) ABSTRACT

The present invention relates to a method for the prevention or treatment of certain breast cancers or ovarian cancer comprising administering to a patient in need thereof of a therapeutically effective amount of a 17,20-lyase inhibitor, wherein the breast cancer or ovarian cancer is estrogen receptor (ER) negative.

### PRIORITY CLAIM

This application claims the benefit under 35 U.S.C. §119(e) of U.S. Provisional Patent Application No. 61/415,097, filed Nov. 18, 2010, hereby incorporated by reference in its entirety.

### FIELD OF THE INVENTION

The present invention relates to a method for the prevention or treatment of certain breast cancers or ovarian cancer comprising administering to a patient in need thereof a therapeutically effective amount of a 17,20-lyase inhibitor.

### BACKGROUND OF THE INVENTION

There are many parallels between prostate cancer and breast cancer, both in underlying disease biology and therapeutic approaches. Like prostate cancer, breast cancer is a steroid-hormone driven neoplastic disease (Jordan V. C. et al., Cancer Res. 2009, 69:1243-54; Folkerd, E. J. and Dowsett, M., J. Clin. Oncol. 2010, 28:4038-44) and it is estimated that 207,000 women in the United States alone will be newly diagnosed with breast cancer this year and that approximately 40,000 women will die from their disease (www.cancer.gov/cancertopics/types/breast; accessed Nov. 5, 2010). When newly diagnosed, breast cancer is predominantly characterized as ‘estrogen dependent’ (over 70% of newly diagnosed BrCa is estrogen receptor (ER) positive; (Foulkes, W. D., et al. Cancer Res. 2004, 10: 2029-34; Williams, M. R. et al., Br. J. Cancer 1987, 55: 67-73) and disease progression and development is closely associated with, and driven by, estrogen-receptor (ER) activity (Jordan V. C. et al., Cancer Res. 2009, 69:1243-54; Folkerd, E. J. and Dowsett, M., J. Clin. Oncol 2010, 28:4038-44; Sanchez, A. M., et al., Molec. Endocrin. 2010, 24: 2114-25). 100041 As disease progresses beyond early-stage localized disease, therapy usually focuses upon disrupting steroid hormone receptor functionality. Current approaches to achieve this rely upon administration of receptor antagonists (i.e., tamoxifen, fulvestrant) either alone or combined with agents that inhibit the production of estrogens, such as aromatase (P-450ₐᵣₒₘ, CYP19) inhibitors (i.e., exemestane, anastrozole or letrozole) (Burstein, H. J. and Griggs, J. J., Surg. Oncol. Clin. N Amer. 2010, 19: 639-47; Burstein, H. J. et al., J. Clin. Oncol. 2010, 28: 3784-96; Stuart-Harris, R. and Davis, A., Womens Health (England) 2010, 6:383-98). Therapeutically, breast cancer therapies that are focused to disrupt ER function by altering ligand binding are usually initially effective and, unlike prostate cancer, can result in long-term disease-free survival (>5 years) (Burstein, H. J. and Griggs, J. J., Surg. Oncol. Clin. N Amer. 2010, 19: 639-47; Burstein, H. J. et al., J. Clin. Oncol. 2010, 28: 3784-96; Stuart-Harris, R. and Davis, A., Womens Health (England) 2010, 6:383-98).

Not surprisingly, however, these therapies do often fail. Mechanistically, and quite analogous to the situation confronted in prostate cancer therapeutics, failure can be a reflection of several biological mechanisms (Zilli, M. et al., Biochim. Biophys. Acta 2009, 1795: 62-81). The ER can mutate and become insensitive to antagonist inhibition, become more promiscuous (Davies, M. P. A. et al., Breast Cancer Res. 2004, 7: R113-18) and utilize other steroids as ligands to drive ER activity (Barone, I. et al., Clin. Cancer Res. 2010, 16:2702-08; Ekena, K. et al., J. Biol. Chem. 1998, 273: 693-99; Nichols, M. and McCarty Jr., K. S. Breast Cancer Res. Treat. 2002, 72: 61-68; Barone, I. et al., Cancer Res. 2009, 69: 4724-32; Barone, I. et al., Oncogene 2010, 29: 2404-14) or be able to undergo ligand-independent activation (Zhang, Q-X. et al., Cancer Res. 1997, 57:1244-49; Tremblay, G. B. et al., Cancer Res. 1998, 58: 877-81). Alternatively, other intracellular signaling pathways, (i.e., cytokine- or growth factor-driven) can phosphorylate, and consequently activate (in the absence of ligand), the ER and induce ER binding to response elements (Yoo, Y. A. et al., Biochim. Biophys. Acta 2008, 1783:438-47; Lee, S. H. and Nam, H. S., Molec. Cells 2008, 26: 286-90; Pradham. M. et al., J. Biol. Chem. 2010, 285: 31100-06). Lastly, resistance to anti-estrogen therapy can be associated with an induction of intratumor (extra-gonadal) estrogen synthesis associated with an induction of aromatase (CYP19) and other steroidgenic enzymes (Brodie, A. et al., Urol. Oncol. 2009, 27: 53-63; Haynes, B. P. et al., Clin. Cancer Res. 2010, 16: 1790-1801; Chanplakorn, N. et al., Breast Cancer Res. Treat. 2010, 120: 639-48; Sasano, H. et al., J. Steroid Biochem. Mol. Biol. 2010, 118:242-45).

The androgen receptor (AR) is a member of the steroid receptor super-family (Fuller, P. J. FASEB J. 1991, 5: 3092-99.) that is expressed in a variety of organs and its function is particularly important in the prostate gland where it drives prostate cancer progression (Knudsen, K. E. and Penning, T. M., Trends Endocrinol. Metab. 2010, 21: 315-24). Like the estrogen receptor, the androgen receptor functions as a transcription factor that modulates the expression of a number of target genes (Lamont, K. R. and Tindall, D. J., Adv. Cancer Res. 2010, 107: 137-62.). Indeed, there is considerable overlap in the biological, molecular and pathological consequences of estrogen receptor and androgen receptor activation (Leav, I. et al., Amer. J. Pathology 2001, 159: 79-92; 32; Vihko, P. et al., Mol. Cell. Endocrinol. 2006, 248: 141-48).

The androgen receptor is also expressed in approximately 70% of breast cancers and can be co-expressed with the estrogen and progesterone receptors (Nahleh, Z. Future Oncol. 2008, 4: 15-21; Birrell, S. N. et al., J. Mammary Gland Biol. Neoplasia 1998, 3: 95-103; Brys, M., Med. Sci. Monit. 2000, 6:433-38; Liao, D.J. and Dickson, R. B., J. Steroid Biochem. Mol. Biol. 2002, 20: 175-89). Biologically, androgen receptors that are expressed on breast cancer cells appear to be functionally active following ligand engagement and impact multiple cellular processes (Liao, D. J. and Dickson, R. B., J. Steroid Biochem. Mol. Biol. 2002, 20: 175-89; Park, S., et al., Ann. Oncol. 2010, 21: 488-92; Gucalp, A and Traina T. A., Cancer J 2010, 16: 62-65). However, the cellular effects of androgen receptor activation in breast cancer cells is not well defined and may be growth inhibitory or growth stimulatory (Nahleh, Z. Future Oncol. 2008, 4: 15-21; Lippman, M. et al., Cancer Res. 1976, 36: 4610-18; Goldenberg, I. S. et al., JAMA 1973, 3: 1267-68; Labrie, F. et al., Endocr. Rev. 2003, 24: 152-82; Dimitrakakis, C. et al., Fertil. Steril. 2002, 77: S26-33). Specifically, high circulating levels of androgens in pre-menopausal women predict reduced incidence of developing breast cancer while in post-menopausal women increased circulating androgens correlates with an increased risk of developing breast cancer (Zumoff, B. et al., Cancer Res. 1981, 41: 3360-3363.; Gordon, G. B. et al., Cancer Res. 1990, 50: 3859-62; Dorgan, J. F. et al., Cancer Epidemiol. Biomarkers Prevention 1997, 6: 177-81).

Thus, androgens appear to be protective in premenopausal women but disease stimulating in post menopausal women (Nahleh, Z. Future Oncol. 2008, 4: 15-21) and treatment with exogenous androgens has been shown to induce tumor regressions in approximately 20% of pre-menopausal women with metastatic breast cancer (Goldenberg, I. S. et al., JAMA 1973, 3: 1267-68).

These conflicting clinical findings as a function of menopausal age have been interpreted to suggest that androgen receptor function in breast cancer is conditioned by the co-expression and functionality of other receptors (i.e., steroid, growth factor, etc.) as well as the availability and utilization of specific receptor (i.e., ER and/or PR), co-activators and co-repressors (Hardin, C. et al., World J. Surg. 2007, 31: 1041-1046; Toth-Fejel, S. et al., Arch. Surg. 2004, 139: 50-54; Hankinson, S. E. and Elissen, A. H., J. Steroid Biochem. Mol. Biol. 2007, 106: 24-30; Somboonporn, W. and Davis, S. R., Maturitas 2004, 49: 267-75; Baglietto, L. et al., Cancer EpidemioL Biomarkers Prev. 2010, 19: 492-502; Magklara, A. et al., Breast Cancer Res. Treat. 2000, 59: 263-70; Vienonen, A. et al., Europ. I EndocrinoL 2003, 148: 469-79; Conde, I. et al., Breast Cancer Res. 2004, 6: R140-48). Along these lines, Nahleh suggests that the heterogeneity of carcinoma cells in terms of steroid receptor distribution may influence the activity of androgens in either a positive or negative manner (Nahleh, Z. Future Oncol. 2008, 4: 15-21).

In general, therapy for progressive, advanced or metastatic breast cancer has focused on inhibition of non-steroid (i.e., growth factor associated) mechanisms of proliferation such as inhibition of EFGR/HER2 activity with targeted agents like trastuzumab or lapatinib and chemotherapeutic agents such as doxorubicin, cisplatin, 5-fluorouracil, cyclophosphamide, or cytarabine. Responses to these therapeutic approaches may be short-lived, resulting in continued disease progression. Triple negative breast cancer is a specific sub-type in which the tumor is estrogen receptor (ER) negative, progesterone receptor (PR) negative and epidermal growth factor receptor-2 (HER2 or HER2/neu negative and is thus difficult to treat with current therapies (Schneider, B. P. et al., Clin. Cancer Res. 2008, 14(24):8010-8018).

It is estimated that 21,880 new cases of ovarian cancer will be diagnosed in the United States in 2010 and that 13,850 people will die from the disease in 2010 (http://www.cancer.gov/cancertopics/types/ovarian; accessed 10 Nov. 2010). Most of the ovarian cancers are ovarian epithelial carcinomas (cancer that begins in the cells on the surface of the ovary) (http://www.cancer.gov/cancertopics/types/ovarian; accessed 10 Nov. 2010). A recent study identified a subgroup of epithelial ovarian cancers which were negative for ER, PR and HER2 expression which accounted for 15.5% of patients studied (Liu et al., J. Clin. Pathol. 2010, 63:240-243). This study also demonstrated that similar to triple-negative breast cancer, most triple negative epithelial ovarian cancers showed a more aggressive clinicopathological phenotype than non-triple negative epithelial ovarian cancers (Liu et al., J Clin. Pathol. 2010, 63:240-243).

Other cancers in which sex hormones may play a role in the progression of the cancer include uterine cancer, endometrial cancer, non-small cell lung cancer and colorectal cancer (Folkerd, E. J. and Dowsett. M., J. Clin. Oncol. 2010, 28: 4038-4044; Paggi M. G. et al., Cancer Lett. 2010, 298(1):1-8; Fucic A. et al., Toxicol. Pathol. 2010, 38(6):849-55; Gambacciani M. et al., Best Pract Res Clin Endocrinol Metab. 2003 17(1):139-47.

Thus, there remains a need to find new treatments for cancers that are ER negative; and especially for breast and ovarian cancers that are ER negative.

### DETAILED DESCRIPTION OF THE INVENTION

### 1. General Description

The present invention relates to a method for the prevention or treatment of certain cancers comprising administering to a patient in need thereof of a therapeutically effective amount of a 17,20-lyase inhibitor.

The present invention also relates to a method for the prevention or treatment of certain breast cancers or ovarian cancer comprising administering to a patient in need thereof of a therapeutically effective amount of a 17,20-lyase inhibitor.

### 2. Definitions

As used herein, the term “cancer” refers to a cellular disorder characterized by uncontrolled or disregulated cell proliferation, decreased cellular differentiation, inappropriate ability to invade surrounding tissue, and/or ability to establish new growth at ectopic sites. The term “cancer” includes, but is not limited to, solid tumors and bloodborne tumors. The term “cancer” encompasses diseases of skin, tissues, organs, bone, cartilage, blood, and vessels. The term “cancer” further encompasses primary and metastatic cancers.

As used herein, the terms HER2 or HER2/neu negative refers to an immunohistochemistry scoring of 0/1+ in laboratory testing as defined by the American Society of Clinical Oncologisₜs (ASCO) and the College of American Pathologists (CAP) and published in Wolff et al., Arch. Path. Lab. Med. 2007, 131 18-43.

As used herein, the term estrogen receptor (ER) negative refers to a finding of <1% of tumor cell nuclei that are immunoreactive in the presence of evidence that the sample can express ER (positive intrinsic controls are seen) as defined by the American Society of Clinical Oncologists (ASCO) and the College of American Pathologists (CAP) and published in Hammond et al., Arch. Path. Lab. Med 2010, 134(6): 907-922.

As used herein, the term progesterone receptor (PR) negative refers to a finding of <1% of tumor cell nuclei that are immunoreactive in the presence of evidence that the sample can express PR (positive intrinsic controls are seen) as defined by the American Society of Clinical Oncologists (ASCO) and the College of American Pathologists (CAP) and published in Hammond et al., Arch. Path. Lab. Med. 2010, 134(6): 907-922.

As used herein, the term “triple negative breast cancer” refers to a breast cancer that is HER2 negative, ER negative and PR negative.

As used herein, the term, “triple negative ovarian cancer” or “triple negative epithelial ovarian cancer” refers to a cancer that is HER2 negative, ER negative and PR negative.

As used herein, the term “CYP17 inhibitor” means an inhibitor of 17,20-lyase which may additionally inhibit 17-alpha hydroxylase.

As used herein, the term “patient” means an animal, preferably a mammal, and most preferably a human.

As used herein, the term “therapeutically effective amount” refers to the amount of the compound, pharmaceutically acceptable salt or pharmaceutical composition that is effective for treating or preventing the disease or disorder.

### 3. Detailed Description

The present invention relates to methods for the prevention or treatment of certain breast cancers or ovarian cancers comprising administering to a patient in need thereof a therapeutically effective amount of a 17,20-lyase inhibitor.

As outlined above, in certain types of breast cancer which are ER negative, androgen receptor function may be responsible for disease progression. Circulating androgens, and in some cases, androgen precursors or ligands can activate the androgen receptor. CYP17 is a key enzyme involved in the biosynthesis of androgens. Thus, inhibition of CYP 17 via inhibition of 17,20-lyase can lead to inhibition of androgen receptor ligand biosynthesis and thus androgen receptor function. As outlined above, in cancers that are ER positive, inhibition of androgen receptor function may enhance tumor growth. However, in cancers that are ER negative, inhibition of androgen receptor function by inhibiting androgen ligand synthesis without necessarily direct inhibition of the androgen receptor may decrease tumor growth.

Thus, there is a new and unexpected use for 17,20-lyase inhibitors in the treatment of those breast and ovarian cancers which are ER negative.

In some embodiments, the invention relates to a method for the prevention or treatment of certain cancers comprising administering to a patient in need thereof a therapeutically effective amount of a 17,20-lyase inhibitor, wherein the cancer is ER negative.

In some embodiments, the invention relates to a method for the prevention or treatment of certain cancers comprising administering to a patient in need thereof a therapeutically effective amount of a 17,20-lyase inhibitor, wherein the cancer is ER negative, and wherein the cancer is breast cancer (male and female), ovarian cancer, uterine cancer, endometrial cancer, non-small cell lung cancer, or colorectal cancer.

In some embodiments, the invention relates to a method for the prevention or treatment of breast cancer or ovarian cancer comprising administering to a patient in need thereof a therapeutically effective amount of a 17,20-lyase inhibitor, wherein the breast cancer or ovarian cancer is ER negative.

In some embodiments, the invention relates to a method for the prevention or treatment of breast cancer or ovarian cancer comprising administering to a patient in need thereof a therapeutically effective amount of a 17,20-lyase inhibitor, wherein the breast cancer or ovarian cancer is ER negative and PR negative.

In some embodiments, the invention relates to a method for the prevention or treatment of breast cancer or ovarian cancer comprising administering to a patient in need thereof a therapeutically effective amount of a 17,20-lyase inhibitor, wherein the breast cancer or ovarian cancer is ER negative, PR negative, and HER-2 negative.

In some embodiments, the invention relates to a method for the prevention or treatment of breast cancer or ovarian cancer comprising administering to a patient in need thereof a therapeutically effective amount of a 17,20-lyase inhibitor, wherein the 17,20-lyase inhibitor is 3β-acetoxy-17-(3-pyridyl)androsta-5,16-diene, 6-[(7S)-7-hydroxy-6,7-dihydro-5H-pyrrolo[1,2-c]imidazol-7-yl]-N-methyl-2-naphthalenecarboxamide, or 3β-hydroxy-17-(1H-benzimidazol-1-yl)androsta-5,16-diene, or pharmaceutically acceptable salts, or pharmaceutical compositions thereof, and wherein the breast cancer or ovarian cancer is ER negative.

In some embodiments, the invention relates to a method for the prevention or treatment of breast cancer or ovarian cancer comprising administering to a patient in need thereof a therapeutically effective amount of a 17,20-lyase inhibitor, wherein the 17,20-lyase inhibitor is 3β-acetoxy-17-(3-pyridyl)androsta-5,16-diene, 6-[(7S)-7-hydroxy-6,7-dihydro-5H-pyrrolo[1,2-c]imidazol-7-yl]-N-methyl-2-naphthalenecarboxamide, or 3β-hydroxy-17-(1H-benzimidazol-1-yl)androsta-5,16-diene, or pharmaceutically acceptable salts, or pharmaceutical compositions thereof, and wherein the breast cancer or ovarian cancer is ER negative and PR negative.

In some embodiments, the invention relates to a method for the prevention or treatment of breast cancer or ovarian cancer comprising administering to a patient in need thereof a therapeutically effective amount of a 17,20-lyase inhibitor, wherein the 17,20-lyase inhibitor is 3β-acetoxy-17-(3-pyridyl)androsta-5,16-diene, 6-[(7S)-7-hydroxy-6,7-dihydro-5H-pyrrolo[1,2-c]imidazol-7-yl]-N-methyl-2-naphthalenecarboxamide, or 3β-hydroxy-17-(1H-benzimidazol-1-yl)androsta-5,16-diene, or pharmaceutically acceptable salts, or pharmaceutical compositions thereof, and wherein the breast cancer or ovarian cancer is ER negative, PR negative, and HER2 negative.

In some embodiments, the invention relates to a method for the prevention or treatment of breast cancer or ovarian cancer comprising administering to a patient in need thereof a therapeutically effective amount of a 17,20-lyase inhibitor, wherein the 17,20-lyase inhibitor is 6-[(7S)-7-hydroxy-6,7-dihydro-5H-pyrrolo[1,2-c]imidazol-7-yl]-N-methyl-2-naphthalenecarboxamide, or a pharmaceutically acceptable salt, or a pharmaceutical composition thereof, and wherein the breast cancer or ovarian cancer is ER negative.

In some embodiments, the invention relates to a method for the prevention or treatment of breast cancer or ovarian cancer comprising administering to a patient in need thereof a therapeutically effective amount of a 17,20-lyase inhibitor, wherein the 17,20-lyase inhibitor is 6-[(7S)-7-hydroxy-6,7-dihydro-5H-pyrrolo[1,2-c]imidazol-7-yl]-N-methyl-2-naphthalenecarboxamide, or a pharmaceutically acceptable salt, or a pharmaceutical composition thereof, and wherein the breast cancer or ovarian cancer is ER negative and PR negative.

In some embodiments, the invention relates to a method for the prevention or treatment of breast cancer or ovarian cancer comprising administering to a patient in need thereof a therapeutically effective amount of a 17,20-lyase inhibitor, wherein the 17,20-lyase inhibitor is 6-[(7S)-7-hydroxy-6,7-dihydro-5H-pyrrolo[1,2-c]imidazol-7-yl]-N-methyl-2-naphthalenecarboxamide, or a pharmaceutically acceptable salt, or a pharmaceutical composition thereof, and wherein the breast cancer or ovarian cancer is ER negative, PR negative, and HER2 negative.

In some embodiments, the invention relates to a method for the prevention or treatment of breast cancer comprising administering to a patient in need thereof a therapeutically effective amount of a 17,20-lyase inhibitor, wherein the breast cancer is ER negative.

In some embodiments, the invention relates to a method for the prevention or treatment of breast cancer comprising administering to a patient in need thereof a therapeutically effective amount of a 17,20-lyase inhibitor, wherein the breast cancer is ER negative and PR negative.

In some embodiments, the invention relates to a method for the prevention or treatment of breast cancer comprising administering to a patient in need thereof a therapeutically effective amount of a 17,20-lyase inhibitor, wherein the breast cancer is ER negative, PR negative, and HER2 negative.

In some embodiments, the invention relates to a method for the prevention or treatment of breast cancer comprising administering to a patient in need thereof a therapeutically effective amount of a 17,20-lyase inhibitor, wherein the 17,20-lyase inhibitor is 3β-acetoxy-17-(3-pyridyl)androsta-5,16-diene, 6-[(7S)-7-hydroxy-6,7-dihydro-5H-pyrrolo[1,2-c]imidazol-7-yl]-N-methyl-2-naphthalenecarboxamide, or 3β-hydroxy-17-(1H-benzimidazol-1-yl)androsta-5,16-diene, or pharmaceutically acceptable salts, or pharmaceutical compositions thereof, and wherein the breast cancer is ER negative.

In some embodiments, the invention relates to a method for the prevention or treatment of breast cancer comprising administering to a patient in need thereof a therapeutically effective amount of a 17,20-lyase inhibitor, wherein the 17,20-lyase inhibitor is 3β-acetoxy-17-(3-pyridyl)androsta-5,16-diene, 6-[(7S)-7-hydroxy-6,7-dihydro-5H-pyrrolo[1,2-c]imidazol-7-yl]-N-methyl-2-naphthalenecarboxamide, or 3β-hydroxy-17-(1H-benzimidazol-1-yl)androsta-5,16-diene, or pharmaceutically acceptable salts, or pharmaceutical compositions thereof, and wherein the breast cancer is ER negative and PR negative.

In some embodiments, the invention relates to a method for the prevention or treatment of breast cancer comprising administering to a patient in need thereof a therapeutically effective amount of a 17,20-lyase inhibitor, wherein the 17,20-lyase inhibitor is 3β-acetoxy-17-(3-pyridyl)androsta-5,16-diene, 6-[(7S)-7-hydroxy-6,7-dihydro-5H-pyrrolo[1,2-c]imidazol-7-yl]-N-methyl-2-naphthalenecarboxamide, or 3β-hydroxy-17-(1H-benzimidazol-1-yl)androsta-5,16-diene, or pharmaceutically acceptable salts, or pharmaceutical compositions thereof, and wherein the breast cancer is ER negative, PR negative and HER2 negative.

In some embodiments, the invention relates to a method for the prevention or treatment of breast cancer comprising administering to a patient in need thereof a therapeutically effective amount of a 17,20-lyase inhibitor, wherein the 17,20-lyase inhibitor is 6-[(7S)-7-hydroxy-6,7-dihydro-5H-pyrrolo[1,2-c]imidazol-7-yl]-N-methyl-2-naphthalenecarboxamide, or a pharmaceutically acceptable salt, or a pharmaceutical composition thereof, and wherein the breast cancer is ER negative.

In some embodiments, the invention relates to a method for the prevention or treatment of breast cancer comprising administering to a patient in need thereof a therapeutically effective amount of a 17,20-lyase inhibitor, wherein the 17,20-lyase inhibitor is 6-[(7S)-7-hydroxy-6,7-dihydro-5H-pyrrolo[1,2-c]imidazol-7-yl]-N-methyl-2-naphthalenecarboxamide, or a pharmaceutically acceptable salt, or a pharmaceutical composition thereof, and wherein the breast cancer is ER negative and PR negative.

In some embodiments, the invention relates to a method for the prevention or treatment of breast cancer comprising administering to a patient in need thereof a therapeutically effective amount of a 17,20-lyase inhibitor, wherein the 17,20-lyase inhibitor is 6-[(7S)-7-hydroxy-6,7-dihydro-5H-pyrrolo[1,2-c]imidazol-7-yl]-N-methyl-2-naphthalenecarboxamide, or a pharmaceutically acceptable salt, or a pharmaceutical composition thereof, and wherein the breast cancer is ER negative, PR negative, and HER2 negative.

In some embodiments, the invention relates to a method for the prevention or treatment of ovarian cancer comprising administering to a patient in need thereof a therapeutically effective amount of a 17,20-lyase inhibitor, wherein the ovarian cancer is ER negative.

In some embodiments, the invention relates to a method for the prevention or treatment of ovarian cancer comprising administering to a patient in need thereof a therapeutically effective amount of a 17,20-lyase inhibitor, wherein the ovarian cancer is ER negative and PR negative.

In some embodiments, the invention relates to a method for the prevention or treatment of ovarian cancer comprising administering to a patient in need thereof a therapeutically effective amount of a 17,20-lyase inhibitor, wherein the ovarian cancer is ER negative, PR negative, and HER2 negative.

In some embodiments, the invention relates to a method for the prevention or treatment of ovarian cancer comprising administering to a patient in need thereof a therapeutically effective amount of a 17,20-lyase inhibitor, wherein the 17,20-lyase inhibitor is 3β-acetoxy-17-(3-pyridyl)androsta-5,16-diene, 6-[(7S)-7-hydroxy-6,7-dihydro-5H-pyrrolo[1,2-c]imidazol-7-yl]-N-methyl-2-naphthalenecarboxamide, or 3β-hydroxy-17-(1H-benzimidazol-1-yl)androsta-5,16-diene, or pharmaceutically acceptable salts, or pharmaceutical compositions thereof, and wherein the ovarian cancer is ER negative.

In some embodiments, the invention relates to a method for the prevention or treatment of ovarian cancer comprising administering to a patient in need thereof a therapeutically effective amount of a 17,20-lyase inhibitor, wherein the 17,20-lyase inhibitor is 3β-acetoxy-17-(3-pyridyl)androsta-5,16-diene, 6-[(7S)-7-hydroxy-6,7-dihydro-5H-pyrrolo[1,2-c]imidazol-7-yl1-N-methyl-2-naphthalenecarboxamide, or 3β-hydroxy-17-(1H-benzimidazol-1-yl)androsta-5,16-diene, or pharmaceutically acceptable salts, or pharmaceutical compositions thereof, and wherein the ovarian cancer is ER negative and PR negative.

In some embodiments, the invention relates to a method for the prevention or treatment of ovarian cancer comprising administering to a patient in need thereof a therapeutically effective amount of a 17,20-lyase inhibitor, wherein the 17,20-lyase inhibitor is 3β-acetoxy-17-(3-pyridyl)androsta-5,16-diene, 6-[(7S)-7-hydroxy-6,7-dihydro-5H-pyrrolo[1,2-c]imidazol-7-yl]-N-methyl-2-naphthalenecarboxamide, or 3β-hydroxy-17-(1H-benzimidazol-1-yl)androsta-5,16-diene, or pharmaceutically acceptable salts, or pharmaceutical compositions thereof, and wherein the ovarian cancer is ER negative, PR negative and HER2 negative.

In some embodiments, the invention relates to a method for the prevention or treatment of ovarian cancer comprising administering to a patient in need thereof a therapeutically effective amount of a 17,20-lyase inhibitor, wherein the 17,20-lyase inhibitor is 6-[(7S)-7-hydroxy-6,7-dihydro-5H-pyrrolo[1,2-c]imidazol-7-yl]-N-methyl-2-naphthalenecarboxamide, or a pharmaceutically acceptable salt, or a pharmaceutical composition thereof, and wherein the ovarian cancer is ER negative.

In some embodiments, the invention relates to a method for the prevention or treatment of ovarian cancer comprising administering to a patient in need thereof a therapeutically effective amount of a 17,20-lyase inhibitor, wherein the 17,20-lyase inhibitor is 6-[(7S)-7-hydroxy-6,7-dihydro-5H-pyrrolo[1,2-c]imidazol-7-yl]-N-methyl-2-naphthalenecarboxamide, or a pharmaceutically acceptable salt, or a pharmaceutical composition thereof, and wherein the ovarian cancer is ER negative and PR negative.

In some embodiments, the invention relates to a method for the prevention or treatment of ovarian cancer comprising administering to a patient in need thereof a therapeutically effective amount of a 17,20-lyase inhibitor, wherein the 17,20-lyase inhibitor is 6-[(7S)-7-hydroxy-6,7-dihydro-5H-pyrrolo[1,2-c]imidazol-7-yl]-N-methyl-2-naphthalenecarboxamide, or a pharmaceutically acceptable salt, or a pharmaceutical composition thereof, and wherein the ovarian cancer is ER negative, PR negative, and HER2 negative.

17,20-lyase Inhibitors

In some embodiments, the 17,20-lyase inhibitor may additionally inhibit 17-alpha hydroxylase. In some embodiments, the 17,20-lyase inhibitor is 3β-acetoxy-17-(3-pyridyl)androsta-5,16-diene, 6-[(7S)-7-hydroxy-6,7-dihydro-5H-pyrrolo[1,2-c]imidazol-7-yl]-N-methyl-2-naphthalenecarboxamide, or 3β-hydroxy-17-(1H-benzimidazol-1-yl)androsta-5,16-diene, or pharmaceutically acceptable salts, or pharmaceutical compositions thereof. In certain embodiments, the 17,20-lyase inhibitor is 3β-acetoxy-17-(3-pyridyl)androsta-5,16-diene, or a pharmaceutically acceptable salt, or a pharmaceutical composition thereof. In certain embodiments, the 17,20-lyase inhibitor is 6-[(7S)-7-hydroxy-6,7-dihydro-5H-pyrrolo[1,2-c]imidazol-7-yl]-N-methyl-2-naphthalenecarboxamide, or a pharmaceutically acceptable salt, or a pharmaceutical composition thereof. In certain embodiments, the 17,20-lyase inhibitor is 3β-hydroxy-17-(1H-benzimidazol-1-yl)androsta-5,16-diene, or a pharmaceutically acceptable salt, or a pharmaceutical composition thereof.

The compound 6-[(7S)-7-hydroxy-6,7-dihydro-5H-pyrrolo[1,2-c]imidazol-7-yl]-N-methyl-2-naphthalenecarboxamide (I) is disclosed in U.S. Pat. No. 7,141,598, and U.S. Appl. Publ. No. 2005/0043544, which are both herein incorporated by reference in their entirety. Methods for the synthesis of the compound 6-[(7S)-7-hydroxy-6,7-dihydro-5H-pyrrolo[1,2-c]imidazol-7-yl]-N-methyl-2-naphthalenecarboxamide are disclosed in U.S. Appl. Publ. No. 2005/0043544, herein incorporated by reference in its entirety. Methods of treatment of androgen independent prostate cancer using 6-[(7S)-7-hydroxy-6,7-dihydro-5H-pyrrolo[1,2-c]imidazol-7-yl]-N-methyl-2-naphthalenecarboxamide and pharmaceutical compositions thereof, are disclosed in Int. App. Pub. No. WO09/057795 which is herein incorporated by reference in its entirety.

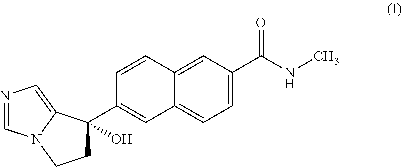

Abiraterone acetate (3β-acetoxy-17-(3-pyridyl)androsta-5,16-diene; IIb) is a pro-drug form of abiraterone (3β-hydroxy-17-(3-pyridyl)androsta-5,16-diene; IIa). Abiraterone acetate is a pharmaceutically acceptable salt of abiraterone. Both abiraterone and abiratetone acetate are disclosed in U.S. Pat. No. 5,604,213 herein incorporated by reference in its entirety, and can by synthesized by methods described in U.S. Pat. Nos. 5,604,213 and 5,618,807, which are both herein incorporated by reference in their entirety. Other pharmaceutically acceptable salts of abiraterone are disclosed in U.S. Pat. No. 7,700,766, which is hereby incorporated by reference in its entirety. Of special interest is abiraterone acetate mesylate salt (3β-acetoxy-17-(3-pyridyl)androsta-5,16-diene mesylate salt).

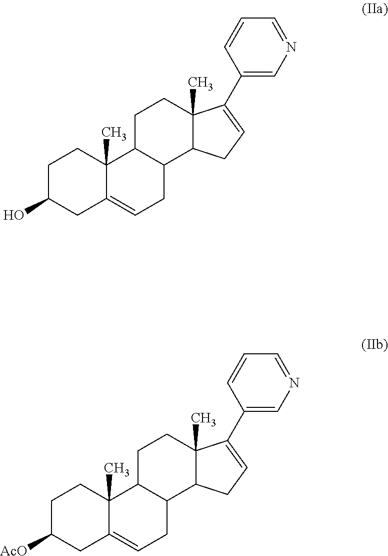

The compound 3β-hydroxy-17-(1H-benzimidazol-1-yl)androsta-5,16-diene (III) is disclosed and methods for its synthesis are described in Intl. App. Pub. No. WO06/093993, which is herein incorporated by reference in its entirety.

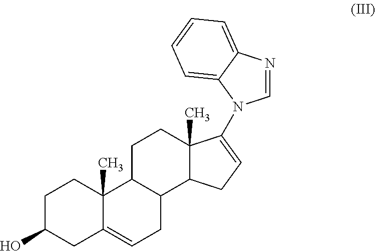

### 4. Formulation and Administration

In another aspect of the present invention, pharmaceutical compositions are provided, wherein these compositions comprise any of the compounds as described herein, and optionally comprise a pharmaceutically acceptable carrier, adjuvant or vehicle. In certain embodiments, these compositions optionally further comprise one or more additional therapeutic agents.

It will also be appreciated that certain of the compounds of present invention can exist in free form for treatment, or where appropriate, as a pharmaceutically acceptable derivative thereof. According to the present invention, a pharmaceutically acceptable derivative includes, but is not limited to, pharmaceutically acceptable prodrugs, salts, esters, salts of such esters, or any other adduct or derivative which upon administration to a patient in need is capable of providing, directly or indirectly, a compound as otherwise described herein, or a metabolite or residue thereof.

As used herein, the term “pharmaceutically acceptable salt” refers to those salts which are, within the scope of sound medical judgement, suitable for use in contact with the tissues of humans and lower animals without undue toxicity, irritation, allergic response and the like, and are commensurate with a reasonable benefit/risk ratio. A “pharmaceutically acceptable salt” means any non-toxic salt or salt of an ester of a compound of this invention that, upon administration to a recipient, is capable of providing, either directly or indirectly, a compound of this invention or an inhibitorily active metabolite or residue thereof. As used herein, the term “inhibitorily active metabolite or residue thereof” means that a metabolite or residue thereof is also an inhibitor of 17,20-lyase.

Pharmaceutically acceptable salts are well known in the art. For example, S. M. Berge et al., describe pharmaceutically acceptable salts in detail in J. Pharmaceutical Sciences, 1977, 66, 1-19, incorporated herein by reference. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium and N⁺(C₁₋₄alkyl)₄ salts. This invention also envisions the quaternization of any basic nitrogen-containing groups of the compounds disclosed herein. Water or oil-soluble or dispersable products may be obtained by such quaternization. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate.

As described above, the pharmaceutically acceptable compositions of the present invention additionally comprise a pharmaceutically acceptable carrier, adjuvant, or vehicle, which, as used herein, includes any and all solvents, diluents, or other liquid vehicle, dispersion or suspension aids, surface active agents, isotonic agents, thickening or emulsifying agents, preservatives, solid binders, lubricants and the like, as suited to the particular dosage form desired. Remington's Pharmaceutical Sciences, Sixteenth Edition, E. W. Martin (Mack Publishing Co., Easton, Pa., 1980) discloses various carriers used in formulating pharmaceutically acceptable compositions and known techniques for the preparation thereof. Except insofar as any conventional carrier medium is incompatible with the compounds of the invention, such as by producing any undesirable biological effect or otherwise interacting in a deleterious manner with any other component(s) of the pharmaceutically acceptable composition, its use is contemplated to be within the scope of this invention. Some examples of materials which can serve as pharmaceutically acceptable carriers include, but are not limited to, ion exchangers, alumina, aluminum stearate, lecithin, serum proteins, such as human serum albumin, buffer substances such as phosphates, glycine, sorbic acid, or potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, polyacrylates, waxes, polyethylene-polyoxypropylene-block polymers, wool fat, sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil; safflower oil; sesame oil; olive oil; corn oil and soybean oil; glycols; such a propylene glycol or polyethylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator.

The compounds and compositions, according to the method of the present invention, may be administered using any amount and any route of administration effective for treating the disease. The exact amount required will vary from subject to subject, depending on the species, age, and general condition of the subject, the severity of the disease, the particular agent, its mode of administration, and the like. The compounds of the invention are preferably formulated in dosage unit form for ease of administration and uniformity of dosage. The expression “dosage unit form” as used herein refers to a physically discrete unit of agent appropriate for the patient to be treated. It will be understood, however, that the total daily usage of the compounds and compositions of the present invention will be decided by the attending physician within the scope of sound medical judgment. The specific effective dose level for any particular patient or organism will depend upon a variety of factors including the disease being treated and the severity of the disease; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed, and like factors well known in the medical arts.

The pharmaceutically acceptable compositions of this invention can be administered to humans and other animals orally, rectally, parenterally, intracisternally, intravaginally, intraperitoneally, topically (as by powders, ointments, or drops), bucally, as an oral or nasal spray, or the like, depending on the severity of the disease being treated. In certain embodiments, the compounds of the invention may be administered orally or parenterally at dosage levels of about 0.01 mg/kg to about 50 mg/kg and preferably from about 1 mg/kg to about 25 mg/kg, of subject body weight per day, one or more times a day, to obtain the desired therapeutic effect.

Liquid dosage forms for oral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a compound of the present invention, it is often desirable to slow the absorption of the compound from subcutaneous or intramuscular injection. This may be accomplished by the use of a liquid suspension of crystalline or amorphous material with poor water solubility. The rate of absorption of the compound then depends upon its rate of dissolution that, in turn, may depend upon crystal size and crystalline form. Alternatively, delayed absorption of a parenterally administered compound form is accomplished by dissolving or suspending the compound in an oil vehicle. Injectable depot forms are made by forming microencapsule matrices of the compound in biodegradable polymers such as polylactide-polyglycolide. Depending upon the ratio of compound to polymer and the nature of the particular polymer employed, the rate of compound release can be controlled. Examples of other biodegradable polymers include poly(orthoesters) and poly(anhydrides). Depot injectable formulations are also prepared by entrapping the compound in liposomes or microemulsions that are compatible with body tissues.

Compositions for rectal or vaginal administration are preferably suppositories which can be prepared by mixing the compounds of this invention with suitable non-irritating excipients or carriers such as cocoa butter, polyethylene glycol or a suppository wax which are solid at ambient temperature but liquid at body temperature and therefore melt in the rectum or vaginal cavity and release the active compound.

Solid dosage forms for oral administration include capsules, tablets, pills, powders, and granules. In such solid dosage forms, the active compound is mixed with at least one inert, pharmaceutically acceptable excipient or carrier such as sodium citrate, calcium dihydrogen phosphate or dicalcium phosphate and/or a) fillers or extenders such as starches, lactose, sucrose, glucose, mannitol, microcrystalline cellulose and silicic acid, b) binders such as, for example, carboxymethylcellulose, hydroxypropylcellulose, alginates, gelatin, polyvinylpyrrolidinone, sucrose, and acacia, c) humectants such as glycerol, d) disintegrating agents such as agar—agar, calcium carbonate, potato or tapioca starch, alginic acid, certain silicates, sodium starch glycolateand sodium carbonate, e) solution retarding agents such as paraffin, 0 absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes. Solid compositions of a similar type may also be employed as fillers in soft and hard-filled gelatin capsules using such excipients as lactose or milk sugar as well as high molecular weight polethylene glycols and the like.

The active compounds can also be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Dosage forms for topical or transdermal administration of a compound of this invention include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, ear drops, and eye drops are also contemplated as being within the scope of this invention. Additionally, the present invention contemplates the use of transdermal patches, which have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

In some embodiments, the method comprises administration of a therapeutically effective amount of a 17,20-lyase inhibitor in combination with an anticancer agent. As used herein, the term “anticancer agent” refers to any agent that is administered to a subject with cancer. Combination therapy includes administration of the therapeutic agents concurrently or sequentially. Alternatively, the therapeutic agents can be combined into one composition which is administered to the patient.

Non-limiting examples of DNA damaging chemotherapeutic agents include topoisomerase I inhibitors (e.g., irinotecan, topotecan, camptothecin and analogs or metabolites thereof, and doxorubicin); topoisomerase II inhibitors (e.g., etoposide, teniposide, and daunorubicin); alkylating agents (e.g., melphalan, chlorambucil, busulfan, thiotepa, ifosfamide, carmustine, lomustine, semustine, streptozocin, decarbazine, methotrexate, mitomycin C, and cyclophosphamide); DNA intercalators (e.g., cisplatin, oxaliplatin, and carboplatin); DNA intercalators, free radical generators such as bleomycin; and nucleoside mimetics (e.g., 5-fluorouracil, capecitibine, gemcitabine, fludarabine, cytarabine, mercaptopurine, thioguanine, pentostatin, and hydroxyurea), estrogen receptor antagonists (e.g. tamoxifen, fulvestrant), and aromatase inhibitors (e.g. exemestane, anastrozole or letrozole)

Chemotherapeutic agents that disrupt cell replication include: paclitaxel, docetaxel, and related analogs; vincristine, vinblastin, and related analogs; thalidomide, lenalidomide, and related analogs (e.g., CC-5013 and CC-4047); protein tyrosine kinase inhibitors (e.g., imatinib mesylate, gefitinib, lapatinib); proteasome inhibitors (e.g., bortezomib); NF-κB inhibitors, including inhibitors of IκB kinase; antibodies which bind to proteins overexpressed in cancers and thereby downregulate cell replication (e.g., trastuzumab, rituximab, cetuximab, and bevacizumab); and other inhibitors of proteins or enzymes known to be upregulated, over-expressed or activated in cancers, the inhibition of which downregulates cell replication.

In some embodiments, the method comprises administration of a therapeutically effective amount of a 17,20-lyase inhibitor in combination with an anticancer agent, wherein the anticancer agent is 5-fluorouracil, cyclophosphamide, doxorubicin, cytarabine, tamoxifen, fulvestrant, cisplatin, exemestane, anastrozole, letrozole, trastuzumab, or lapatinib.

### 4. Experimental Procedures

### Cell Lines and Reagents

The cells lines used in the experiments are a panel of human breast tumor cell lines that express the ER, PR, AR and erbB family receptors (HER2 and EGFR). These cell lines are purchased through the American Type Culture Collection (ATCC) and their growth conditions and growth characteristics have been well defined. The cell lines include the following: MCF-7; T47D; BT-474; MDA-MD 435; MDA-MB-231; 465 and MDA-MB-453.

Specific growth media, media supplements, fetal bovine serum, dialysed fetal bovine serum (MW cutoff of 10,000) and charcoal stripped serum is purchased from Gibco/Invitrogen. Steroid supplements, such as steroid hormones (i.e., estrogen, testosterone, DHT, etc.), steroid precursors (i.e., pregnenolone, androstenediol, estrone, etc) are purchased from either Sigma or Gibco/Invitrogen.

### Assay to Determine of Steroidogenesis Capability of Cell Lines

To determine the intracellular steroid biosynthetic capacity of the cell lines, cells are incubated in media containing various steroids and/or steroid precursors. Following predetermined incubation periods (24 hours-120 hours) media is removed and processes to quantify concentrations of steroids by multiple appropriate techniques (i.e., LC/MS, clinical assay kits, etc.) to quantify/monitor cellularsteroid biosynthesis capacity are performed as described in Locke, J. A. et al., Cancer Res. 2008, 68: 6407-6415, and Hofland, J. et al., Cancer Res. 2010, 70: 1256-64.

This assay determines the intracellular capacity of the cell lines to generate androgen molecules from precursors and demonstrates the impact of extracellular androgens on cancer cell proliferation by modulating the concentration of androgens in serum using dialyzed or charcoal-stripped serum. The conditions employed mimic the pharmacological effects of systemic 17,20-lyase inhibition and consequently extracellular androgens are undetectable. Cell lines that are ER positive show minimal amounts of inhibition of proliferation, whereas in cell lines that are ER negative, proliferation is inhibited.

Assay to Determine Impact of a 17,20-lyase Inhibitor on Intracellular Steroid Synthesis

In cell lines that possess the biochemical capacity to generate steroid molecules intracellularly, the effect of a 17,20-lyase inhibitor on intracellular steroid synthesis in cells is determined by incubating cells in media containing the 17,20-lyase inhibitor at final concentrations of 0.001 μM to 100 μM. Following predetermined incubation periods (24 hours-120 hours) the media is removed and processes to quantify concentrations of steroids as described above are performed.

Results from this assay demonstrate that in cells possessing the capacity to generate steroid molecules intracellularly exposure to a 17,20-lyase inhibitor results in inhibition of proliferation demonstrating that AR function in ER negative cells or cell ines can be targeted by reduction of androgen biosynthesis using a 17,20-lyase inhibitor.

### Assay to Determine Cytotoxic Effects of Anticancer Drugs Under Conditions of Androgen Deprivation (Drug Combination Studies)

To determine the impact of reduced extracellular androgens on the cytotoxic activity of anticancer agents, cells are cultured in media supplemented with either charcoal-stripped serum or dialysed serum (with or without steroids or steroid precursors) in a 96 well plate. After 24 hours a 17,20-lyase inhibitor alone (0.001 μM-100 μM) or in combination with a second anticancer agent (0.001 μM-100 μM) is added to each well. After an additional 72 hours cell viability/proliferation in each well is assessed by either the MTT or APT-lite assays, as described below. Wells containing DMSO alone serve as blanks and wells containing cells alone serve as positive controls. Data generated in these assays is assessed by conventional fractionated dose analysis as described in Darnowski, J. W. et al., Biochem. Pharmacol. 1997, 53: 571-80.

### Assays to Determine Cell Viability/Proliferation

Cell viability and proliferation is assessed by monitoring mitrochondrial function using the mitochondrial dye 3-[4,5-dimethyltiazol-2-yl]-2,5-diphenyl-tetrazoliumbromide (MTT). Following incubation in media alone or media supplemented with various steroids or test compounds over a range of concentrations, 30 μl of 5 mg/ml MTT is added to each well. After an additional 4 hours incubation at 37° C. 100 μl of buffered DMSO is added to each well and absorbance is determined at 570 nm with a BIO-RAD 550 microplate reader. Wells containing DMSO alone serve as blanks and well containing cells alone serve as positive controls (see Darnowski, J. W. et al., Cancer Chemother. Pharmacol. 2004, 54: 249-58; Darnowski, J. W. et al., J. Biol. Chem. 2006, 281:17707-17).

Alternatively, cell viability is assessed using the ATP-lite kit assay (Perkin-Elmir) to quantify cellular ATP content. In this assay cells incubated in media alone or supplemented with various steroids or inhibitory compounds are processed exactly as described in the manufacturer's instructions. ATP content correlates with cellular luminescence as determined by luminometer (Wallac) analysis.

What is claimed is:

1. A method for the prevention or treatment of breast cancer or ovarian cancer comprising administering to a patient in need thereof a therapeutically effective amount of a 17,20-lyase inhibitor, wherein the breast cancer or ovarian cancer is ER negative.

2. The method of claim 1, wherein the breast or ovarian cancer is ER negative and PR negative.

3. The method of claim 1, wherein the breast or ovarian cancer is ER negative, PR negative, and HER2 negative.

4. The method of any of claims 1-3, wherein the 17,20-lyase inhibitor is 3β-acetoxy-17-(3-pyridyl)androsta-5,16-diene, 6-[(7S)-7-hydroxy-6,7-dihydro-5H-pyrrolo[1,2-c]imidazol-7-yl]-N-methyl-2-naphthalenecarboxamide, or 3β-hydroxy-17-(1H-benzimidazol-1-yl)androsta-5,16-diene, or pharmaceutically acceptable salts, or pharmaceutical compositions thereof.

5. The method of any of claims 1-3, wherein the 17,20-lyase inhibitor is 6-[(7S)-7-hydroxy-6,7-dihydro-5H-pyrrolo[1,2-c]imidazol-7-yl]-N-methyl-2-naphthalenecarboxamide, or a pharmaceutically acceptable salt, or a pharmaceutical composition thereof.

6. A method for the prevention or treatment of breast cancer comprising administering to a patient in need thereof a therapeutically effective amount of a 17,20-lyase inhibitor, wherein the breast cancer is ER negative.

7. The method of claim 6, wherein the breast cancer is ER negative and PR negative.

8. The method of claim 6, wherein the breast is ER negative, PR negative, and HER2 negative.

9. The method of any of claims 6-8, wherein the 17,20-lyase inhibitor is 3β-acetoxy-17-(3-pyridyl)androsta-5,16-diene, 6-[(7S)-7-hydroxy-6,7-dihydro-5H-pyrrolo[1,2-c]imidazol-7-yl]-N-methyl-2-naphthalenecarboxamide, or 3β-hydroxy-17-(1H-benzimidazol-1-yl)androsta-5,16-diene, or pharmaceutically acceptable salts, or pharmaceutical compositions thereof.

10. The method of any of claims 6-8, wherein the 17,20-lyase inhibitor is 6-[(7S)-7-hydroxy-6,7-dihydro-5H-pyrrolo[1,2-c]imidazol-7-yl]-N-methyl-2-naphthalenecarboxamide, or a pharmaceutically acceptable salt, or a pharmaceutical composition thereof.

11. A method for the prevention or treatment of ovarian cancer comprising administering to a patient in need thereof a therapeutically effective amount of a 17,20-lyase inhibitor, wherein the ovarian cancer is ER negative.

12. The method of claim 11, wherein the ovarian cancer is ER negative and PR negative.

13. The method of claim 11, wherein the ovarian is ER negative, PR negative, and HER2 negative.

14. The method of any of claims 11-13, wherein the 17,20-lyase inhibitor is 3β-acetoxy-17-(3-pyridyl)androsta-5,16-diene, 6-[(7S)-7-hydroxy-6,7-dihydro-5H-pyrrolo[1,2-c]imidazol-7-yl]-N-methyl-2-naphthalenecarboxamide, or 3β-hydroxy-17-(1H-benzimidazol-1-yl)androsta-5,16-diene, or pharmaceutically acceptable salts, or pharmaceutical compositions thereof.

15. The method of any of claims 11-13, wherein the 17,20-lyase inhibitor is 6-[(7S)-7-hydroxy-6,7-dihydro-5H-pyrrolo[1,2-c]imidazol-7-yl]-N-methyl-2-naphthalenecarboxamide, or a pharmaceutically acceptable salt, or a pharmaceutical composition thereof.

16. The method of any of claim 1-3, 6-8 or 11-13, comprising administering to a patient in need thereof a therapeutically effective amount of an anticancer agent in combination with the 17,20-lyase inhibitor.

17. The method of claim 16, wherein the anticancer agent is 5-fluorouracil, cyclophosphamide, doxorubicin, cytarabine, tamoxifen, fulvestrant, cisplatin, exemestane, anastrozole, letrozole, trastuzumab, or lapatinib.
